(19) United States
(12) Patent Application Publication
(10) Pub. No.: US 2018/0177453 A1
(43) Pub. Date: Jun. 28, 2018

(54) MEDICATION OR TREATMENT MONITORING DEVICE

(71) Applicants: Maria Stephanie R. JARDELEZA, San Antonio, TX (US); Henrick Youval KRIGEL, San Antonio, TX (US)

(72) Inventors: Maria Stephanie R. JARDELEZA, San Antonio, TX (US); Henrick Youval KRIGEL, San Antonio, TX (US)

(73) Assignee: THE BOARD OF REGENTS OF THE UNIVERSITY OF TEXAS SYSTEM, Austin, TX (US)

(21) Appl. No.: 15/735,994

(22) PCT Filed: Jun. 17, 2016
(86) PCT No.: PCT/US2016/037987

§ 371 Date: Dec. 13, 2017

Related U.S. Application Data
(60) Provisional application No. 62/181,094, filed on Jun. 17, 2015.

(51) Int. Cl. A61B 5/00; A61B 5/11; A61F 9/00

(52) U.S. Cl. CPC A61B 5/4833; A61B 5/1114; A61B 5/0022; A61B 5/6831; A61B 5/742; A61B 5/6814; A61F 9/0008

(57) ABSTRACT

Certain embodiments are directed to a device alerts patients to when an eye drop is due and whether an eye drop has already been administered. Other embodiments include a device that helps the patients to keep their heads tilted at certain positioning after a retinal tear or detachment repair. The device provides feedback to the ophthalmologist on the patient's compliance with the eye drops regimen. The device provides a direct measure of how well the patient adheres to the eye drops required by the physician. This allows the ophthalmologist to adequately assess whether a medical regimen or surgical regimen is failing due to patient compliance or treatment failure.

This Application claims priority to U.S. Provisional Patent Application Ser. No. 62/181,094 filed Jun. 17, 2015, which is incorporated herein by reference in its entirety.

### BACKGROUND

The present invention relates to methods and devices for monitoring medication or treatment schedules.

It is conventional for a doctor to prescribe one or more medications for a patient to take one or more times during the day, and perhaps at certain times of the day. The medications are usually in the form of pills but may also be powders and liquids. Often more than one medication must be taken on a daily basis. Various devices have been provided to make it easier for patients to organize their daily medication. One of these devices is in the form of a container having seven different compartments, each for a different day of the week. Room is provided in each compartment for one or more pills that the patient must take on that day.

Such a device does not provide any indication on when the medication contained in a particular compartment must be taken, particularly if the administration of such medication is time sensitive and is administered in conjunctions with other medications in a particular temporal regime. If multiple medications must be administered at different times during the day or in a particular order, the presence of several medications for a given day does not give the patient any information other than that medication must be taken during a particular day.

In a variety of situations, such as after repair of a retinal tear or detachment a patient is provided a number of medications to be administered in particular sequences with a particular delay between administrations. Furthermore, patients that undergo surgery for a retinal tear or detachment are often required to keep their heads tilted at a certain positioning for prolonged periods to help with healing and recovery of the retina. Use of eye drops at regular intervals for several weeks after surgery for retinal tear or detachment, or any other ocular surgery is required to minimize the risk of post-surgical complications. The number and frequency of eye drops frequently change as patients proceed with treatment. In addition, patients with chronic eye conditions requiring prolonged and multiple eye drop use, such as for patients with glaucoma and ocular inflammation, require strict compliance with eye drops regimen to preserve vision. Inability to comply with eye drops use at appropriate time intervals can lead to disease progression and blindness. Thus, there is a need for additional devices and methods for monitoring medication and treatments for complex treatment regimes.

### SUMMARY

Certain embodiments are directed to a monitoring device comprising a microprocessor, a display, a user interface, a clock, and data storage. In certain aspects the device is configured as a wearable device. In a further aspect the device is configured to be worn on the wrist, wrist arm, or waist; or carried or kept in the proximity of a patient. The device comprises a user interface that can include a touch screen, push button, or switch for entering data. In certain embodiments the data storage is EEprom data memory. The device is programmed to monitor one or more medication or treatment schedules. In certain aspects the medication schedule is an eye drop schedule. The user interface can provide for data entry, such as confirmation that a schedule task, treatment, or medication has been administered. In certain aspects the device will display the schedule by type of medication or treatment, and the time the medication or treatment is to be applied or administered.

In certain respects the device can be programmed with 1, 2, 3, 4, 5 or more medication or treatment schedules. Each schedule is programmable on a per patient basis. The schedule can comprise type of medication(s), dosage(s), and timing of application or administration. The management of multiple schedules on one device can help in minimizing drug interactions.

In certain aspects the device is configured to record user input/compliance over a period of time. The data can be stored locally on the device for a period time until downloaded or transmitted to a receiving device or server. The device can be configured to transmit data when a communication connection is available. The device can be connected to a network The device can be configured to store data locally until a communication connection is available, e.g., SD memory card. In certain aspects the data can be stored on a removable storage that is periodically removed, data downloaded, and re-inserted.

Certain embodiments are directed to additional devices for monitoring patients and the positioning of all or part of the patient's body. In certain aspects a head position device is used to monitor the positioning of patient's head. In other aspects the device can be a limb (leg or arm) position device as well as a torso position device—the position devices described are used to determine the angles or position of the target body or body part. In certain aspects a head position sensor is worn on the head. In certain aspects the head position device is in communication with a microprocessor based display and data logging unit and a data storage device. Alternative embodiments include sensors mounted in a wristband, legband, or waistband. In certain aspects the display/data logging device is configured as a wrist wearable device. In certain embodiments the data storage device is EEprom data memory. After setting the head in the desired position, the head position is registered electronically, and the values of the three axis are saved in the unit's memory for reference.

In certain embodiments a positioning device and a monitoring device can be configured to share or use the same user interface or display.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of monitoring device and head position monitor.

FIG. 2. Schematic of one example of a monitoring device.

FIG. 3. One example of a routine for monitoring application of eye drops or other medications.

FIG. 4. One example of a routine for programming sequence display controlled by micro-processor.

FIG. 5 One example of a routine for initiating a head position monitoring aspect of the method described.

FIG. 6 One example of a routine for displaying head position as it relates to the X-axis.

FIG. 7 One example of a routine for displaying head position as it relates to the X-axis.

### DESCRIPTION

Embodiments are directed to a device and system for monitoring compliance with treatment or medication regimes. A treatment of medical regime can be prescribed by a physician or other qualified medical personnel. The patient can be provided a description of the treatment or medication and specific instructions concerning each medication. In certain aspects unique identifiers for the medication can be provided. Unique identifiers can include, but are not limited to containers, stickers, caps, labels, geometric shapes, etc. that can be used to distinguish one medication from another. Each medication can be associated with a particular unique identifier, e.g., number, label, or color. In certain aspects the device is programmed to use the unique identifier, e.g., color, number, or label, to identify a particular medication.

For the purpose of this disclosure, the term “medication” is used to include medicines in tablet, powder, liquid, ointment or any other form representing discrete dosages to be taken at least once, and generally, at periodic intervals during the day or during a number of days.

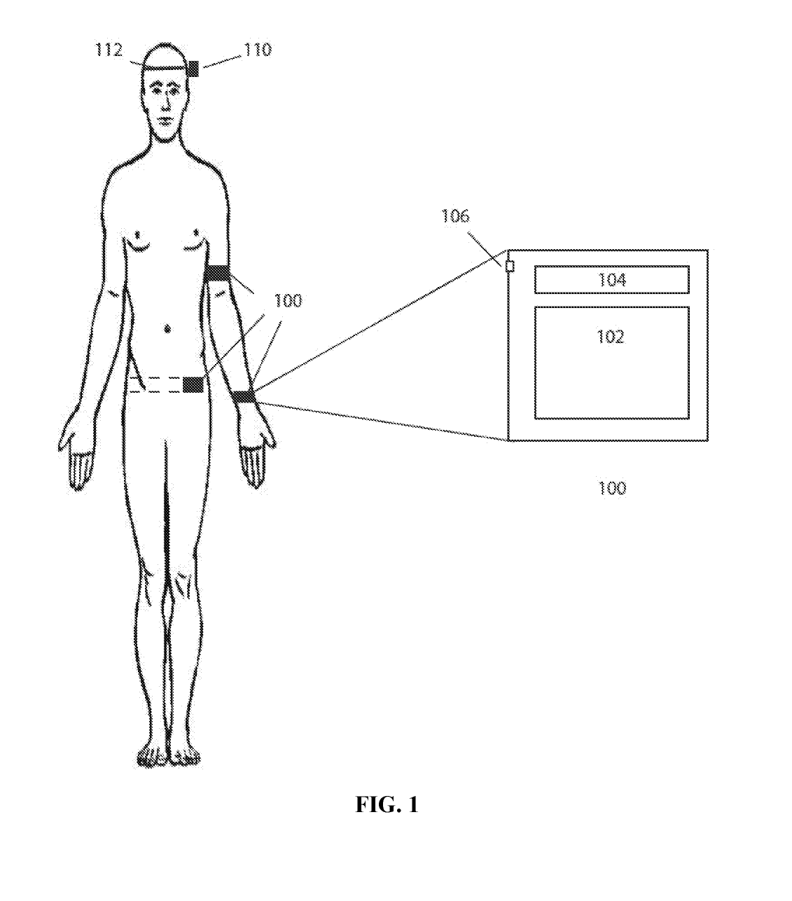

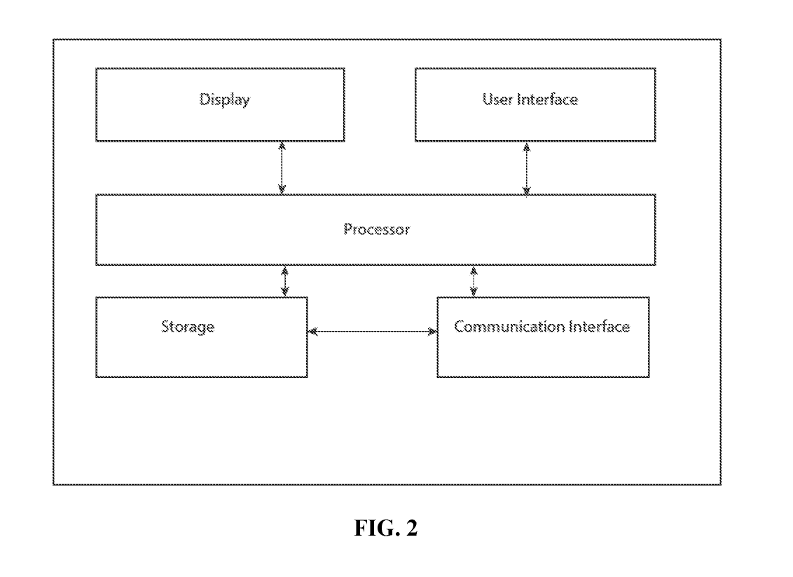

In certain respects and referring to FIG. 1 and FIG. 2, monitoring device 100 comprises a processor, data storage, a display, a user interface and optionally a communication interface. The device can be configured as a wearable device. The device can be worn attached to the patient's wrist, neck, arm, waist, etc. (see FIG. 1). The device can have display 102, alphanumeric window 104, and push button 106 (used as part of the user interface). The communication interface can accommodate one or more of wired communication interfaces (USB, microUSB, etc.) or wireless communication interfaces (WiFi, Bluetooth, etc.). In certain aspects the user interface can include a touch screen or scroll device. In certain aspects an smartphone, smartwatch, tablet, or other mobile device can be configured or programmed to integrate various aspects of the methods and process described herein. In certain aspects a mobile application or software can be used to implement and/or control the methods and devices described herein.

The device can be programmed with a multiplicity of medication schedules. The device can be provided to a patient along with a multiplicity of unique identifiers that can be associated with medications. A database having entries representing different medications and schedules can be queried and the appropriate records transmitted and stored on the device. Once the appropriate data is installed on the device the medical care professional can modify parameters as needed for a patient—for example a physician programs a particular medicine at a particular dose or amount to be administered, or a particular treatment to be applied on a particular schedule.

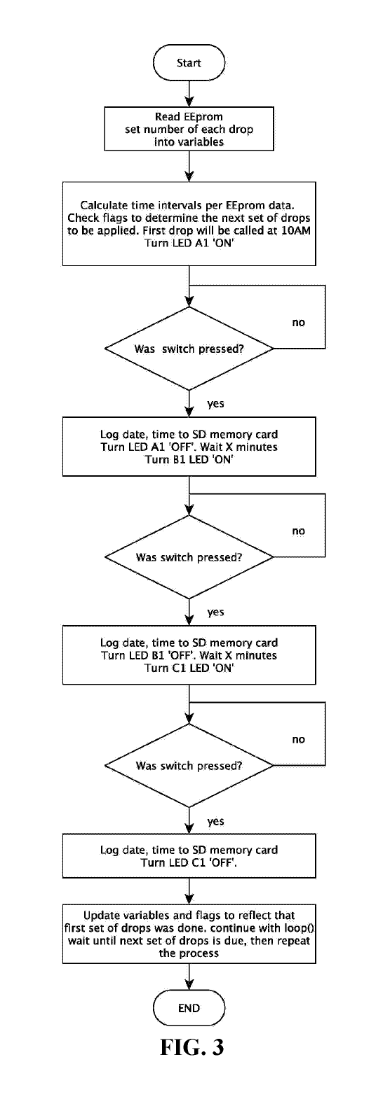

FIG. 3 illustrates an example flow chart for monitoring application of eye drops, for an example. In this example a microprocessor is programmed for a sequence of three drops. To initiate the process the processor queries storage to set the number of each drop into the appropriate variable. The program calculates the time intervals per the prescribed schedule. The program identifies the medication to be administered and determines the time for administration, for example first drop is due for administration at 10:00 am. The program activates an indicator that the first drop is due. If the user activates the user interface and indicates the drop was administered then the date and time are recorded and the indicator that a drop is due is inactivated. After a predetermined wait time the indicator for drop due is activated again, indicating a second drop is due. If the user interface is activated then the drop has been administered and the time and date recorded. The drop due indicator is turned off and a predetermined period of time is allowed to pass, when the third drop is needed the indicator will be activated. If the user interface is activated then the drop has been administered and the indicator light can be inactivated. Once the series of drops are completed the variables and schedule program are updated to reflect that this series of drops was completed, and setting the time for the next administration and repeating the process. In certain aspects each administration can be the same or a different medication that is administered with a predetermined time between administrations. The time can be at least 0.2, 0.5, 1, 5, 10, 15 or more minutes.

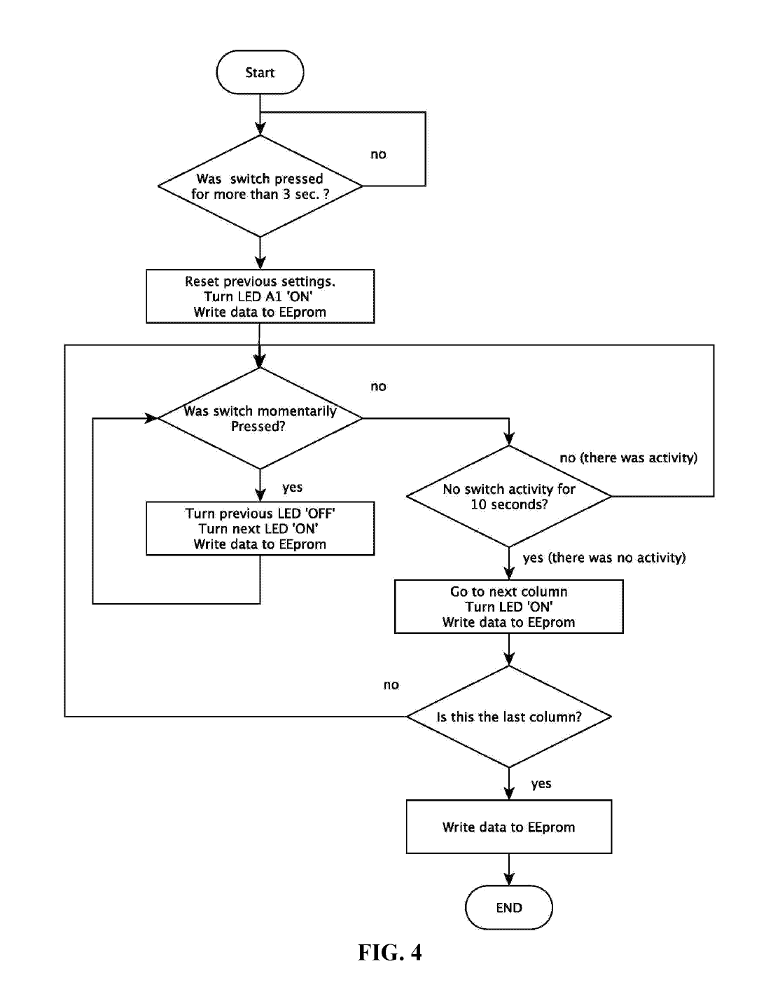

FIG. 4 illustrates an example of a control process for a programming a device described herein. To initiate one aspect of the programming a qualified or authorized medical user activates the interface for at least three seconds. In response to the continuous activation for three seconds the device resets and displays activation of the first scheduled task. Activation of a task can be indicated by turning on an LED. Upon activation of the user interface during the programming period a second indicator is activated to indicate the first task is complete and the second task is due. This loop is continued for each medication until the last column is activated. Once the last column is activated the schedule is complete, data is transmitted to storage. The routine can be initiated at the start of each medication or treatment cycle, which can be selected from 1, 2, 3, 4, 5, 6, 7, 8, 9, 10 or more days, weeks, or months.

In certain aspects a display or speaker can provide visual or audible instructions or indicator, for example, instructions to the patient concerning the number of tablets or drops that should be taken by the patient. In certain aspects information is provided in alphanumeric form across or in a text window of the device. In a further aspect information is provided in a display window. The display can be configured to provide graphics. Still other aspects the display can be a row or an array light emitting diodes or other light sources. In certain embodiments the display can provide visual or audio signals related to an upcoming task (e.g., reminders), completion of a task (e.g., providing positive reinforcement), failure to complete tasks (e.g., warnings or alerts), etc. In certain aspects a signal can be communicated to a server that is monitored by a third party, e.g., nurse or staff at a physician's office or hospital.

At the time a task is scheduled the patient can receive a visual or audio indicator that a medicine or treatment needs to be administered or applied. In certain aspects the visual or audio indicator correlates with a unique identifier for the medicine or treatment. For example if a particular medication is due and the medication bottle has a unique identifier that is blue (e.g., a blue cap) the device will display a blue color, the word “blue”, or will say “blue”. Thus, informing the patient that it is time to administer or apply the medication or treatment associated with the blue unique identifier.

In certain aspects the device is capable of displaying the types of medications, and the times of the day a patient needs to take or administer certain medications or treatments. The administration or treatment will be determined and prescribed by a physician or other qualified medical personnel. In certain aspects the qualified medical personnel can program or have the device programmed for 1, 2, 3, 4, 5, or more schedules. Every time the patient completes a task, e.g., a displayed treatment or administration, he or she confirms completion of the task by activating a user interface of the device. The user interface can be a touch screen, push button, lever switch, or the like. Activation of the user interface records the event along with the date, the time, and the specific type of medication or treatment via the processor into data storage. The completion or lack of completion of a task will be used to update the status of the device. In certain aspects the status of the device will be displayed periodically. For example, after completion of a task the display can shut off or go dark until time to display the next task(s). This process repeats itself throughout the day based on the schedule(s) programmed. In further aspects the device cycles on a 24-hour clock and starts a new cycle every 24 hours.

When the patient visits the physician, the data in storage can be retrieved, e.g., the device may be plugged into a computer. The physician then can see all the recorded data and may save the data to the patient's medical file. The data shows the physician if the patient followed the directions and helps the physician to evaluate the patient condition.

In certain embodiments the display of the device is arranged in a matrix of LED's. The columns of the matrix represent a medication type, and the rows represent the amount medication that the patient needs to take. The display can be a regular array of LEDs, but is not limited to a regular array of LED's, and may include other types of displays, such as LCD screen or external display such as a smart phone communicating with the device using technologies such as blue-tooth.

In certain embodiments the scheduling device can be coupled to other sensors or monitors. In one aspect the scheduling device is coupled to a head position monitor. The head position monitor is used to help a patient maintain a particular head position and to record the changes in head position over time. Patients that undergo surgery for a retinal tear or detachment are often required to keep their heads tilted at a certain positioning for prolonged periods to help with healing and recovery of the retina. Keeping the head positioned at the exact angle required by the surgeon is crucial for patient's recovery and surgical success. The exact angle of the head position is determined by the ophthalmologist based on the location of the retinal tear or retinal detachment. Practically, it is difficult for the patient to replicate the exact head position outside the physician's office. In addition, there is no way for the ophthalmologist to objectively measure compliance with the correct position aside for the patient's self reported compliance. As a result, should the patient require another surgery for a retinal re-detachment, it cannot be correctly attributed to surgical technique or patient compliance. In certain embodiments a head position monitor can be programmed to indicate when the head is positioned appropriately. Indicators can be provided in a treatment or medication scheduling device to provide feedback to the position and to assist in maintaining the appropriate head position. Lights on the coupled scheduling device can be used to indicate when the head is in the appropriate position, e.g., by showing a green light associated with a head position indicator, or when the head is in an inappropriate position, e.g., displaying read lights, or when the head is somewhere in between, e.g., displaying yellow lights. The indicator lights can be display indicative of the x, y and z axis, which will assist the patient in orienting head position. The coupled schedule device can also monitor and record head position over time when the patient is not in the presence of a health care professional.

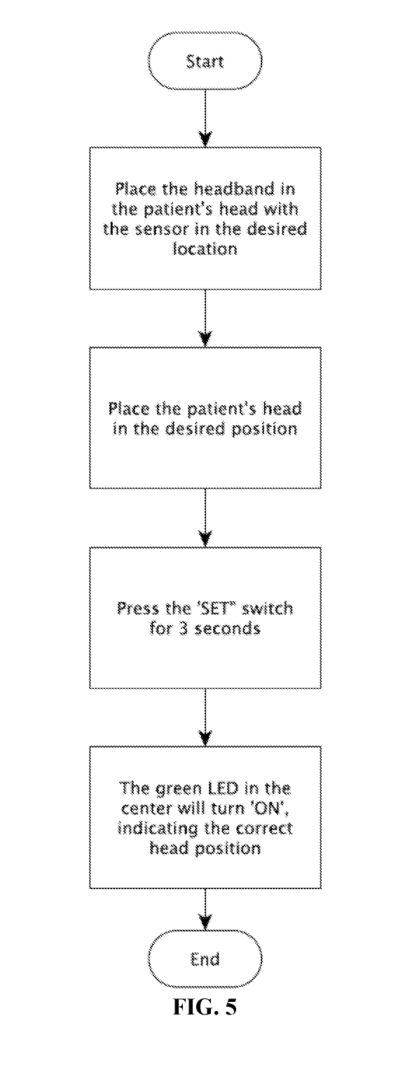

FIG. 5 provides a flow chart describing one example of initiating the head positioning monitor. First a headband or other attachment device comprising the sensor is attached to the patient's head and the sensor positioned in a desired location. The patient's head is then positioned in the desired position. The monitor, sensor, or monitor and sensor are set to the desired position. Setting of the monitor or device can be accomplished by pressing and holding a ‘SET’ switch for a pre-determined time. In certain aspect an indicator, such as a green indicator light can inform the person setting the monitor and/or sensor the set-up is complete. Other methods or routines can be used to initiate or set the desired head position of the patient.

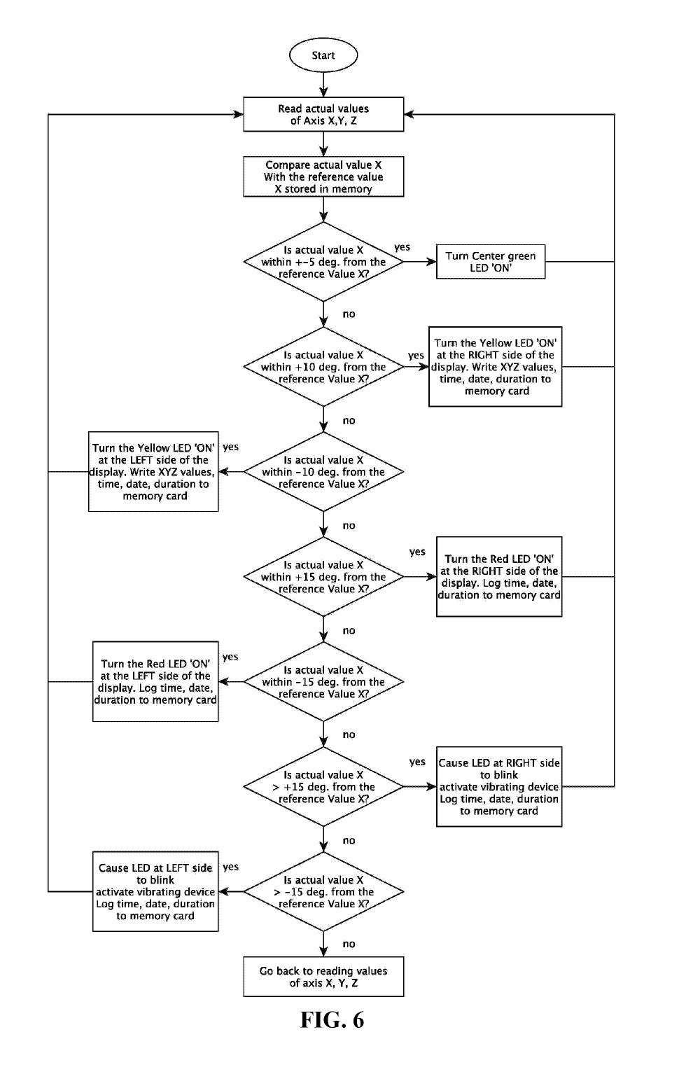

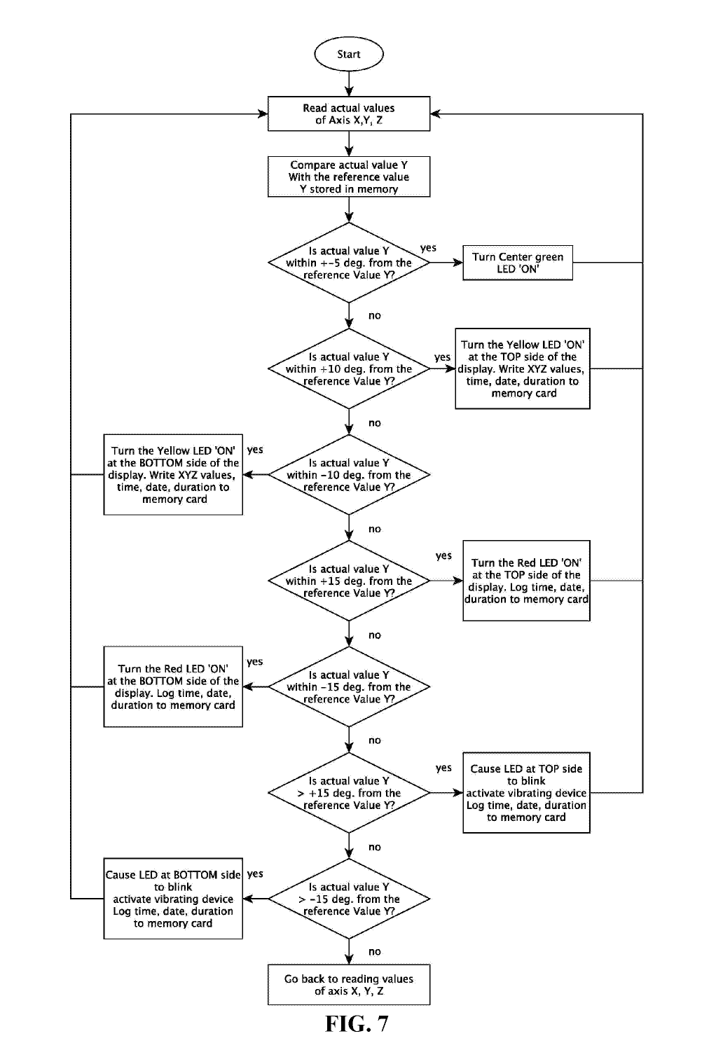

FIG. 6 and FIG. 7 illustrate an example of a routine for displaying a head position in the x or y axis, respectively. In certain aspects the device will monitor head position and provide audio indicator (e.g., beep), visual indicator (e.g., lights), tactile indicator (e.g., vibration) or a combination thereof, the indicator relating to the relationship between the current head position and the initial head position setting. In other aspects the indicators can indicate a good head position or one that is within a range of acceptable positions relative to the initial head position, a bad head position or one that may be detrimental to the treatment outcome, or an intermediate head position in that is a position that is acceptable but not ideal in regard to fostering a positive treatment outcome. The routines charted in FIG. 6 and FIG. 7 query the head position and respond with an appropriate indicator, which is provided as a visual indicator in the example provided. In the example provided a head position within 5 degrees of the initial setting is a good head position, a head position within 10 degrees and greater than 5 degrees of the initial head position is an intermediate position, and a head position within 15 degrees and greater than 10 degrees is a bad position. Any position over 15 degrees will trigger an elevated response to bring the head positions to the patient's or medical personnel's attention. The device will log various parameters associated with each head position query. The parameters can include, but are not limited to head position, time, duration, etc. to a memory device or transmit to server.

In certain aspects a head position monitor can use a three-axis accelerometer to detect head position and movement. In other aspects the sensor can be configured to detect trauma to the head or acute changes in head position that can lead to adverse surgical outcomes, such as fall or accidents. An accelerometer is an electromechanical sensor that can detect and/or measure physical acceleration experienced by an object. Thus an accelerometer can measure any movement or vibration of an object. There are many different types of accelerometers depending on the ways acceleration is measured. Piezoelectric accelerometers contain microscopic piezoelectric crystals. Piezoelectric elements can induce voltage when a pressure is applied. Thus if acceleration forces causes any stress or pressure on the microscopic piezoelectric crystals acceleration can be measured from the voltage generated. Capacitive accelerometers sense a change in electrical capacitance, with respect to acceleration. These accelerometers may have structures with certain capacitance between them. If an accelerative force moves one of the structures, then the capacitance will change. A 3 axis accelerometer can measure 3 dimensional positioning or movement of an object.

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The matrix of LED's has 8 columns and 8 rows, marked as A, B, C, D, E, F, G, H and 1, 2, 3, 4, 5, 6, 7, 8 respectively. Each column represents the eye-drop or medication type. For example: A=eye-drop Fred Forte (white cap), B=eye-drop Ketorolac (grey cap), C=eye-drop Predlisolone (red cap). Each row represents one eye-drop or medication the patient needs to take.

Programming the Eye-Drop/Medication Monitoring and recording device

| | Type | Type | Type | Type | Type | Type | Type | Type |
|---|---|---|---|---|---|---|---|---|
| | A | B | C | D | E | F | G | H |
| Drop/Med | LED | LED | LED | LED | LED | LED | LED | LED |
| #1 | | | | | | | | |
| Drop/Med | LED | LED | LED | LED | LED | LED | LED | LED |
| #2 | | | | | | | | |
| Drop/Med | LED | LED | LED | LED | LED | LED | LED | LED |
| #3 | | | | | | | | |
| Drop/Med | LED | LED | LED | LED | LED | LED | LED | LED |
| #4 | | | | | | | | |
| Drop/Med | LED | LED | LED | LED | LED | LED | LED | LED |
| #5 | | | | | | | | |
| Drop/Med | LED | LED | LED | LED | LED | LED | LED | LED |
| #6 | | | | | | | | |
| Drop/Med | LED | LED | LED | LED | LED | LED | LED | LED |
| #7 | | | | | | | | |
| Drop/Med | LED | LED | LED | LED | LED | LED | LED | LED |
| #8 | | | | | | | | |

Programming the eye-drop/medication monitoring and recording device is shown in FIG. 3. The programming is started by pressing the eye-drop switch mounted on the device, for about 3 seconds. This action resets the previous settings and allows the new settings. Initially, the first LED in column A will turn on. If eye-drop or medication of type A is desired, the eye-drop switch is pressed momentarily. The first LED turns off and the second LED of column A turns on. If type A eye-drop #2 is needed, it is pressed momentarily again to accept drop #2. The process repeats until no more drops of type A are needed. Then after waiting for 10 seconds (without pressing the switch) the first LED of the next column, type B, turns on. The process here is similar to the process described for Eye-drop/Medication of type A. If no drop of a particular type of Drop/Med is required, on waiting for 10 seconds after the LED of that column turns on, the LED of the next column turns on.

By going through the process, the system records the data into the internal EEprom memory, and the microprocessor uses the data to calculate and display the correct types of drops (or medications), the correct numbers of each and the correct time of the day for each desired eye-drop or medication.

1. A device for monitoring medication and treatment regimes comprising a wearable device having a processor, a display, a clock, and storage, wherein the processor is configured to (i) receive one or more medication or treatment schedules, (ii) display a schedule by type of medication or treatment, and the time the medication or treatment is to be applied or administered, and (iii) receive patient input regarding administration of medication or application of treatment.

2. The device of claim 1, further comprising a head position monitor in communication with the device of claim 1.

3. A method of monitoring patient compliance with a medication or treatment schedule comprising:
(a) programming a wearable device configured to display a medication or treatment schedule and provide the patient with user interface to record administration or application of a medication or treatment;
(b) recording administration or application information to storage on the device over a period of time;
(c) transmitting the stored data to a server; and
(d) displaying a representation of the stored data for assessment by a third party.

4. The method of claim 3, further comprising recording data generated by a head position monitor that is communication with the wearable device.
